(19) United States
(12) Patent Application Publication
(10) Pub. No.: US 2025/0339059 A1
(43) Pub. Date: Nov. 6, 2025

(54) Implantable Sensor with Optimal Electrode Distance

(71) Applicant: D.T.R. Dermal Therapy Research Inc., London (CA)

(72) Inventors: Stig Ollmar, Huddinge (SE); Saul Alejandro Rodriguez Duenas, Järfälla (SE); Ulrik Birgersson, Stockholm (SE); Eric Raquette, Stockholm (SE)

(21) Appl. No.: 18/854,465

(22) PCT Filed: Apr. 4, 2023
(86) PCT No.: PCT/EP2023/058749

§ 371 Date: Oct. 4, 2024

(30) Foreign Application Priority Data
Apr. 5, 2022 (EP) 22166766.0

(51) Int. Cl. A61B 5/145; A61B 5/00

(52) U.S. Cl. CPC A61B 5/14532; A61B 5/14503; A61B 5/6847; A61B 5/7271; A61B 2560/0219; A61B 2560/0468; A61B 2562/166

(57) ABSTRACT

A sensor includes a housing, current electrodes for injecting current into tissue, and voltage electrodes for measuring impedance in the tissue. The voltage electrodes are arranged separately from the current electrodes and in between the current electrodes. A coil powers the sensor via a power source. A circuit board is arranged within the housing. The circuit board is electrically connected to the voltage electrodes, the current electrodes, and the coil. The coil is arranged on top of the circuit board. A communication unit is configured for transferring and receiving data packages and is connected to the circuit board. The housing includes the voltage electrodes and the current electrodes on an outer surface. The sensor includes a ferrite sheet arranged in between the coil and the circuit board. The ferrite sheet has the same or a larger extension as the coil as seen in a plane defined by the circuit board.

### CROSS REFERENCE TO RELATED APPLICATIONS

This application is a § 371 national stage of international application no. PCT/EP2023/058749, filed on Apr. 4, 2023, which claims priority to European application no. 22166766.0, filed on Apr. 5, 2022, the entire contents of both of which are hereby incorporated by reference herein.

### FIELD OF THE DISCLOSURE

The invention relates to the field of implantable sensors for electrical measurements in the body. In particular, the invention relates to implantable sensors for the determination of glucose concentration that apply four-point measurement for the determination of impedance in the body tissue, which can be muscle tissue.

### BACKGROUND

Known sensors for implanting in the body typically contain a printed circuit board, a power source and electrodes or chemical electrodes for determining various parameters in the body. Many known sensors have the aim to detect rejection of an organ after organ transplantation by measuring the trend of the impedance in the transplanted organ after transplantation. A sign of organ rejection is an increase (trend) in electric impedance in an organ (organ tissue) after transplantation. Once such an increase started, a doctor can apply drugs to a certain extent, but if a threshold is reached then the organ will be rejected and basically die within the recipient body. Such a sensor is for instance illustrated in the U.S. Pat. No. 5,970,986 B1.

In the U.S. Pat. No. 5,970,986 B1 an apparatus for rejection diagnostics after organ transplantations, which is equipped with an extra corporal base station comprising a high frequency transmitter and receiver unit that can transmit data to an implantable rejection sensor, is disclosed. The implantable rejection sensor comprises an integrated circuit component, thus an IC component, to which a receiving- and transmitting coil are allocated. The implantable rejection sensor further comprises a sensor arrangement comprising of four electrodes arranged on the plane surface of the housing. U.S. Pat. No. 5,970,986 B1 further states that the electrodes and the coils can be integrated in the IC component. The implantable rejection sensor is configured to be fixed onto a donor organ.

It is herewith important to note that the sensor disclosed in U.S. Pat. No. 5,970,986 B1 is for measuring organ rejection and when doing so the parameter that matters is the trend of the impedance in the organ as explained above. The absolute values of impedance are not critical when detecting organ rejection.

The present disclosure however relates to the determination of glucose levels or glucose concentration in body fluids or tissue of a human or animal and thus a living being by correlating measured impedance values in the body fluid or tissue to a database containing a matrix having values of impedance values correlated to glucose levels or glucose concentration.

When glucose concentration is determined in the tissue/body of a living being, which can be summarized with the term “living beings,” the absolute impedance values are of interest since the absolute impedance values correlate to glucose concentration. If these measured values are false then the correlated glucose values are false, which can lead to unwanted consequences when administering insulin.

One issue that falsifies the measured impedance once a sensor is implanted in the body of a living being, is body liquid that collects around certain areas around the implanted sensor and around the electrodes. This body liquid can be blood, wound liquid or other extra cellular liquids or a mixture thereof. Since the electric conductivity is better in liquids, these body liquids affect and falsify the measured impedance and therefore affect the accuracy of the measurement. Tests and reference measurements have shown that body liquids anywhere around the implanted sensor affect the accuracy of the impedance measurements substantially.

The sensor shown in the U.S. Pat. No. 5,970,986 B1 has exactly this problem due to its sharp edges and flat surfaces. However, since U.S. Pat. No. 5,970,986 B1 is concerned with detecting organ rejection, this problem is not mentioned in the document.

Another problem of many known sensors is that the coil for receiving the magnetic field or electromagnetic field from a power source for powering the sensor is arranged on, under or above the printed circuit board (PCB) of the sensor. Since the PCB typically contains conducting loops and the like, the magnetic field induces Eddy Currents in the PCB. Eddy Currents can affect the measurement performance and disturb the energy or power transfer from the power source to the sensor by reducing the allowable or workable distance between the implanted sensor and the external reader and power source, respectively.

Still another issue of known prior art solutions is that the measurement of the impedance needs to be performed in healthy tissue. Typically, when sensors are implanted in the body of a living being, scar tissue will develop around the sensor once all the wounds are healed. This scar tissue is however not representative of the impedance in healthy tissue, since there is less blood flow in the scar tissue. Likewise other body liquids will only enter the scar tissue via diffusion. Therewith it is of importance to go past or outside of this scar tissue when measuring the impedance with an implanted sensor. In other words, the impedance should be measured in healthy tissue, since impedance values measured in the scar tissue are not representative of body fluid parameters.

### SUMMARY

In view of the above it is an object of the present invention to provide an improved sensor for determining glucose concentration in a living being, so that the impedance measurement within tissue of a living being where the sensor is implanted is exact and can be correlated to a database containing glucose concentrations correlated to reference values of impedance.

It is another object of the invention to provide a sensor that is reliable and safe.

It is a further object of the invention to provide a sensor that is comfortable to use for a patient.

In view of the above-mentioned problems and objects the inventors of the present invention have discovered that the thickness of scar tissue in the body of a living being, for example a human, is normally about 2 mm and that it develops at least more or less homogenously around the implanted sensor once the wounds from the implantation of the sensor have healed. Depending on this thickness of the scar tissue, the inventors discovered that the spacing of the current injecting electrodes has to be in a range of 5 mm to 12 mm, preferably about 6 to 10 mm and more preferably about 7 to 9 mm, whilst the voltage sensing electrodes are placed in between the injecting electrodes and at least more or less on a straight line. The voltage sensing electrodes may also be positioned outside the straight line between the current injection electrodes, e.g. in the corners of a square or rectangle.

In a second aspect the inventors of the present invention have realized that it is possible to arrange the coil that is used to power the implanted sensor via an external device using a pulsed or varying magnetic field, outside of the outer housing of the implantable sensor and connect the coil via a conductor to the electronics, thus the circuit board, of the sensor. This means that the coil can be positioned close to the skin of the patient for easy powering during glucose level determination, while the sensor can be placed a bit deeper in the body of the living being. This can improve measurement results, as explained later herein, and protect the sensor better at the same time.

In a third aspect the inventors of the present invention have realized that using a ferrite sheet or magnetic shield between the coil and the circuit reduces induced currents and therewith the occurrence of Eddy Currents in the circuit board during powering of the sensor. The ferrite sheet or magnetic shield has the further advantage that it protects the sensing and injecting electrodes from any magnetic field or electric disturbances during measuring of the impedance, especially if the sensing—and injecting electrodes are positioned on a side of the ferrite sheet that is directed away from the coil. The inventors of the present invention have further realized that it is possible to use a flexible part of a circuit board to connect the coil to the circuit board and to connect the sensing—and injecting electrodes to the circuit board. A further advantage of using a ferrite sheet or magnetic shield in the sensor is that the magnetic field or magnetic flux that is originating from an external device for powering the sensor, is concentrated in the ferrite sheet or magnetic shield and therewith such a magnetic field can be guided towards the coil for better efficiency and therewith improved energy transfer.

The above-mentioned distance between the current injecting electrodes has the effect that the current trajectories that occur during current injection by the current injection electrodes extend beyond the scar tissue. As long as the distance is more than 4 mm the current trajectories will extend beyond the scar tissue. However, there are also limitations to the maximal distance, since the greater that distance is the more will body movements of the living being and inhomogeneous tissue affect measurement results. This means that the optimal distance is rather limited and it has been shown by experiments and testing that the above described and claimed range herein improves measurement results substantially.

Scar tissue herein refers to the build of hardened, fibrous, and not well-bled through tissue that forms when healthy tissue is destroyed by disease, injury or surgery. It has been discovered that scar tissue not only is formed on the surface of a body of an animal or human but also within the body, when implanting a sensor or the like. Such scar tissue is usually formed after 2 (two) to 4 (four) weeks and as mentioned in detail herein about 2 mm in thickness arranged surrounding such a sensor.

Many leading companies in the area of implants and measuring of parameters within the body have and had big issues with their implantable sensors as these sensors typically work for 2 to 4 weeks, thus prior to the buildup of scar tissue, and then cease to deliver useful measurement results due to the developed scar tissue. In many cases such sensor cannot even measure parameters at all after the two-to-four-week period since the measurement process craves exposure to fresh and healthy tissue, body liquid or interstitial fluid, which does not run through scar tissue. This is a problem for optical sensors, thus implantable sensors that use optics to determine parameters. This is also a problem for reagent-based glucose devices because they must always receive a fresh and abundant supply of blood or interstitial fluid. To overcome the scar tissue barrier issue, the herein present sensor works exactly in the opposite manner, it does not deliver stable results prior to the buildup of scar tissue and thus for about two to four weeks. After this period and thus after buildup of scar tissue the herein disclosed sensor provides stable and useful electric impedance values that can be used for glucose determination, basically as long as the sensor stays implanted. The reason for this is the measurement of impedance is beyond the scar tissue due to the correct distance between current injecting electrodes, as disclosed herein.

The inventors of the present invention have further realized that it is necessary to space the voltage sensing electrodes about 1 mm to 4 mm, preferably about 2-3 mm from the current injecting electrodes for optimal measuring results beyond the scar tissue. The current injecting electrodes and the voltage sensing electrodes may be positioned, at least more or less, on a straight line, or sideways from a straight line, displaced versus or in line with the straight line.

This spacing relating to the measurement electrodes is optimal in order to make sure that the current injection from the current injecting electrodes is not affecting the measurement and that the impedance can be measured over a reasonable distance so that a feasible value can be returned. The closer the voltage sensing electrodes are positioned to one another, the less voltage signal would be detected, which would reduce the signal to noise ratio, something that is undesirable. The signal to noise ratio should always be high to have a high-quality signal.

According to a first aspect, disclosed herein is an implantable glucose sensor for determining impedance in tissue of a living being and correlating the measured impedance with known glucose concentrations and impedance values of a database, the implantable glucose sensor comprises:
a housing,
two current injecting electrodes for injecting current into the tissue,
two voltage sensing electrodes for measuring impedance in the tissue, whereby the two voltage sensing electrodes are arranged separately from the two current injecting electrodes,
a coil for powering the implantable glucose sensor via a power source,
a circuit board arranged within the housing, the circuit board being electrically connected to the two voltage sensing electrodes and the two current injecting electrodes and the coil,
a communication unit for transferring and receiving data packages, the communication unit being connected to the circuit board and whereby,
the housing comprises the two voltage sensing electrodes and the two current injecting electrodes on an outer surface.

In the implantable sensor a distance (d) between the two current injecting electrodes on the outer surface is in a range from 5 mm to 12 mm, preferably about 6 to 10 mm and more preferably about 7 to 9 mm.

The above specified sensor and current injecting electrode distancing improves the measurement results and ensures that the impedance in healthy undamaged tissue is measured and only minimally diluted or affected by the impedance of scar tissue around the implanted sensor. The scar tissue is not as well-nourished with body liquids and blood instead it is only nourished by diffusion and therewith the impedance values from scar tissue are not representative as accurate values. The healthy tissue however is representative of impedance values since body liquids and blood is flowing through this tissue unhindered.

In an embodiment the relationship between the distance (d) between the two current injecting electrodes and a distance (e) between one of the two voltage sensing electrodes and a closest current injecting electrode and a distance between the other of the two voltage sensing electrodes and a closest current injecting electrode, respectively follows the equation:

$$e < d / 2 and e \geq 1 mm .$$

Following the above equation will provide a good outcome for the impedance measurement, as explained later herein.

In an embodiment the voltage sensing electrodes may be arranged symmetrically in between the injecting electrodes and wherein a distance (d) between one of the two voltage sensing electrodes and a closest current injecting electrode and a distance (d′) between the other of the two voltage sensing electrodes and a closest current injecting electrode 4′ is in a range from 1 mm to 4 mm, preferably about 2 mm to 3 mm.

Such a distancing between the two current injecting electrodes and the two voltage sensing electrodes improves the measurement since the voltage sensing electrodes do not pick up ill-defined values from the immediate neighborhood of the injecting electrodes due to distorted injected current density and since the distance between the two voltage sensing electrodes is still reasonable for measuring impedance.

In an embodiment the two current injecting electrodes further comprise a first active area made of conductive material and wherein the voltage sensing electrodes comprise a second active area made of conductive material.

The size of the first active area ensures that enough amperage or voltage can be transferred into the tissue so that a high-quality measurement can be achieved, while keeping the current density in the tissue low enough to avoid non-linear effects.

In another embodiment the first active area and the second active area may be made of gold, or other bio-compatible conductive material.

In still another embodiment the two current injecting electrodes may have a longitudinal shape, such as for example an elliptic longitudinal shape or a rectangular longitudinal shape.

In another embodiment the housing may be shaped harmonic and rounded off and convex on a bottom side and on a top side. In another embodiment the housing may be shaped half-convex with at least the side comprising the current injecting electrodes and the voltage sensing electrodes on a convex shaped side.

In another embodiment the housing may be half-convex whereby the current injecting electrodes and voltage sensing electrodes are arranged or positioned on a flat part of the housing.

The housing may be of an ellipsoid shape.

This may reduce any accumulation of liquids around the implantable sensor.

In still another embodiment the implantable sensor may have outer dimensions of the implantable glucose sensor corresponding to a length of 10 mm to 50 mm, preferably 20 mm to 30 mm, a width of 5 mm to 25 mm, preferably 11 mm to 15 mm and a thickness of 1 mm to 15 mm, preferably 2 mm to 5 mm.

The implantable sensor may be an implantable glucose sensor.

According to a second aspect of the invention, disclosed herein is further an implantable sensor for determining impedance in tissue of a living being, the implantable sensor comprising:
a housing,
two current injecting electrodes for injecting current into the tissue, two voltage sensing electrodes for measuring impedance in the tissue,
whereby the two voltage sensing electrodes are arranged separately from the two current injecting electrodes,
a coil for powering the implantable glucose sensor via a power source,
a circuit board arranged within the housing, the circuit board being electrically connected to the two voltage sensing electrodes and the two current injecting electrodes and the coil,
a communication unit for transferring and receiving data packages, the communication unit being connected to the circuit board, and whereby,
the housing comprises the two voltage sensing electrodes and the two current injecting electrodes on an outer surface.

The coil may be connected to the circuit board, the two voltage sensing electrodes, the two current injecting electrodes and the circuit board by conductors.

In an embodiment the coil may be arranged separately from the housing or outside the housing.

In still another embodiment the connector that connects the coil with the circuit board, the two voltage sensing electrodes and the two current injecting electrodes may be a flexible cable.

In still another embodiment the implantable sensor may comprise elements for fastening the coil to tissue of the living being.

The above-described embodiment of the invention improves the energy transfer from an external device and a coil of the external device, respectively, to the coil of the implantable sensor. This means that the coil of the sensor can be positioned or sewn in the tissue right under the skin (or fur) or at least close to the skin so that the coil of the implantable sensor is at least more or less parallel to an outer surface of the skin. Since the coil is rather small, in a range of about 6 mm to 15 mm in diameter, preferably 7 mm to 12 mm in diameter and more preferably about 8 mm to 10 mm it is rather simple to sew the coil close to the skin.

Having the possibility to place the implanted sensor further away from its coil solves two issues, namely:
that during the powering of the coil via the external device, Eddy Currents can basically not affect the other electronic components of the sensor, such as the circuit board, the powering and communication unit, the two voltage sensing and the two current injecting electrodes, and
no energy is lost by induced Eddy Currents produced by the pulsed or varying magnetic field through the coil, and
that the alignment between the coil of the external device and the coil of the implantable sensor is simplified.

According to a further embodiment according to the disclosure herein, the implantable sensor may be an implantable glucose sensor and wherein a glucose concertation in the tissue is determined by correlating a measured impedance to a database containing impedance values and corresponding glucose concentrations.

For an improved alignment according to item iii) above it might further be conceivable to mark the position of the coil of the implantable sensor under the skin with a tattoo or a sticker on the skin so that the patient knows exactly where to put the external device when doing a measurement.

The above-described solutions and embodiments referring to the distancing and positioning of the current injecting electrodes and the voltage sensing electrodes may also be combined in one single solution of an implantable sensor. Thus, the external coil of the implantable sensor may be used with the specified electrode spacing or alternatively it may be used separately.

According to a third aspect of the present invention an implantable sensor for determining glucose level based on electric impedance in tissue of a living being is herewith disclosed, the implantable sensor comprising:
a housing,
two current injecting electrodes for injecting current into the tissue,
two voltage sensing electrodes for measuring impedance in the tissue, whereby the two voltage sensing electrodes are arranged separately from the two current injecting electrodes and in between the current injecting electrodes,
a coil for powering the implantable glucose sensor via a power source,
a circuit board arranged within the housing, the circuit board being electrically connected to the two voltage sensing electrodes and the two current injecting electrodes and the coil, the coil being arranged on top of the circuit board,
a communication unit for transferring and receiving data packages, the communication unit being connected to the circuit board, and
the housing comprising the two voltage sensing electrodes and the two current injecting electrodes on an outer surface.

The implantable sensor further comprises a ferrite sheet arranged in between the coil and the circuit board, whereby the ferrite sheet has the same or a larger extension as the coil as seen in a plane defined by the circuit board.

The implantable sensor according to the above may be configured to measure impedance in tissue and therewith determine glucose concentration in the tissue by correlating the measured impedance values to reference values that are representing known glucose concentrations.

The advantage of an implantable sensor comprising a ferrite sheet or magnetic shield is that the power transfer is improved from an external device, that the implantable sensor can be built in more compact and therewith smaller size and that no Eddy Currents are induced into the circuit board.

In an embodiment a distance between the two current injecting electrodes on the outer surface is in a range from 5 mm to 12 mm, preferably about 6 to 10 mm and more preferably about 7 to 9 mm.

This may ensure that the impedance sensing occurs in healthy tissue as the current trajectories and therewith the equipotential impedance lines extend beyond the scar tissue.

In a further embodiment the ferrite sheet may be arranged on one side of the circuit board and the two current injecting electrodes and the two voltage sensing electrodes may be arranged on an opposite side of the circuit board.

The effect of the above described build up is that no Eddy Currents or other electric or magnetic disturbances affect the voltage sensing—and injecting electrodes, as they are shielded from the coil by the ferrite sheet, especially when the implantable sensor is used during powering.

In still another embodiment the circuit board may comprise at least two flexible integrated parts, a first flexible integrated part for connecting the coil to the circuit board and a second flexible integrated part for connecting the current injecting electrodes and the voltage sensing electrode to the circuit board.

Using such flexible parts of the circuit board simplifies the build up of the sensor and increases reliability as no electric wires, for example copper wires or the like, need to be connected to the circuit board.

In an embodiment a communication circuit may be integrated in the circuit board or it may be arranged separately from the circuit board.

Each and every aspect or feature that relates to one of the three aspects according to the above may be combined with any or alle of the three explained aspects of the invention.

### Explanation of Terms

Below certain expressions and terms used herein are explained and defined.

### Distance

A distance in the context herein as measured on the housing of the sensor is to be interpreted as a distance between two items, objects or points measured on an outer surface of the housing, even if such a housing is convex or even slightly convex.

### Range

The term range herein means that the starting number and the ending number given in the range are included in the range. Further the term mm herein refers to Millimeter.

### Bio-Compatible Conductive Material

The term bio-compatible conductive material herein refers to any bio-compatible conductive material or alloy, such as Gold, Titan, Platinum and so on or any alloy thereof.

### Symmetric or Symmetrically

The term symmetric or symmetrically or symmetry herein refers to any symmetry along a center point or center line. It may thus include bilateral symmetry or 3-dimensional symmetry versus a plane that extends through a center of an arrangement, in the present case of the two current injecting electrodes and the two voltage sensing electrodes. The voltage sensing electrodes and the current injecting electrodes may further be arranged along a straight line on the housing of the sensor or they may at least be arranged in one single plane, or e.g. in the corners of a square or rectangle.

### Ferrite Sheet or Magnetic Shield

Herein the term ferrite sheet or magnetic shield refers to a sheet made of magnetic materials with a thickness of between 0.1 mm to 0.6 mm. The magnetic material influences the magnetic field as it has a higher magnetic permeability than air or tissue and therewith also reduced losses. The ferrite sheet may thus influence and facilitate the direction of the magnetic field herein. Since the magnetic material attracts the magnetic field such magnetic material is also well suitable for magnetic shielding purposes. The ferrite sheet described herein may be glued, taped or bonded to the circuit board and the coil. Further the ferrite sheet may be made of dielectric-magnetic sheet made of a polymer comprising ferrite powder. Alternatively, the ferrite sheet may be made of flexible sintered ferrite sheet material having a high permeability.

Generally, all terms used in the claims are to be interpreted according to their ordinary meaning in the technical field, unless explicitly defined otherwise herein. All references to “a/an/the element, apparatus, part, half, component, means, device, sensor and so on are to be interpreted openly as referring to at least one instance of the element, apparatus, part, half, component, means, device, sensor and so on unless explicitly stated otherwise. The steps of any method disclosed herein do not have to be performed in the exact order disclosed, unless explicitly stated.

### BRIEF DESCRIPTION OF THE DRAWINGS

The present invention will now be described, for exemplary purposes, in more detail by way of an embodiment(s) and with reference to the enclosed drawings, in which:

FIG. 1a schematically illustrates a perspective view of an ellipsoidal implantable glucose sensor;

FIG. 1b schematically illustrates a perspective view similar as FIG. 1, illustrating a distance (d) between the current injecting electrodes well illustrated;

FIG. 2a schematically illustrates a perspective view of an embodiment similar as the one shown in FIG. 1a;

FIG. 2b schematically illustrates a perspective view of an embodiment similar as the one shown in FIG. 2a, illustrating a distance (d′) between the current injecting electrodes;

FIG. 3 schematically illustrates a graphic representation of current trajectories when current is injected in organic tissue via a pair of current injecting electrodes;

FIG. 4 schematically illustrates the interior of an implantable sensor with certain parts omitted for illustrative purposes;

FIG. 5 schematically illustrates the powering of the implantable sensor of FIG. 2a during measuring of the impedance;

FIG. 6 schematically illustrates another embodiment of the sensor according to the invention having a coil arranged outside a housing of the sensor;

FIG. 7 schematically illustrates a similar view as FIG. 5 but with another embodiment of the implantable sensor; and

FIG. 8 schematically illustrates a similar embodiment as the one shown in FIG. 7 illustrating a cross sectional view of an implantable sensor but with a flat side in the housing.

### DETAILED DESCRIPTION

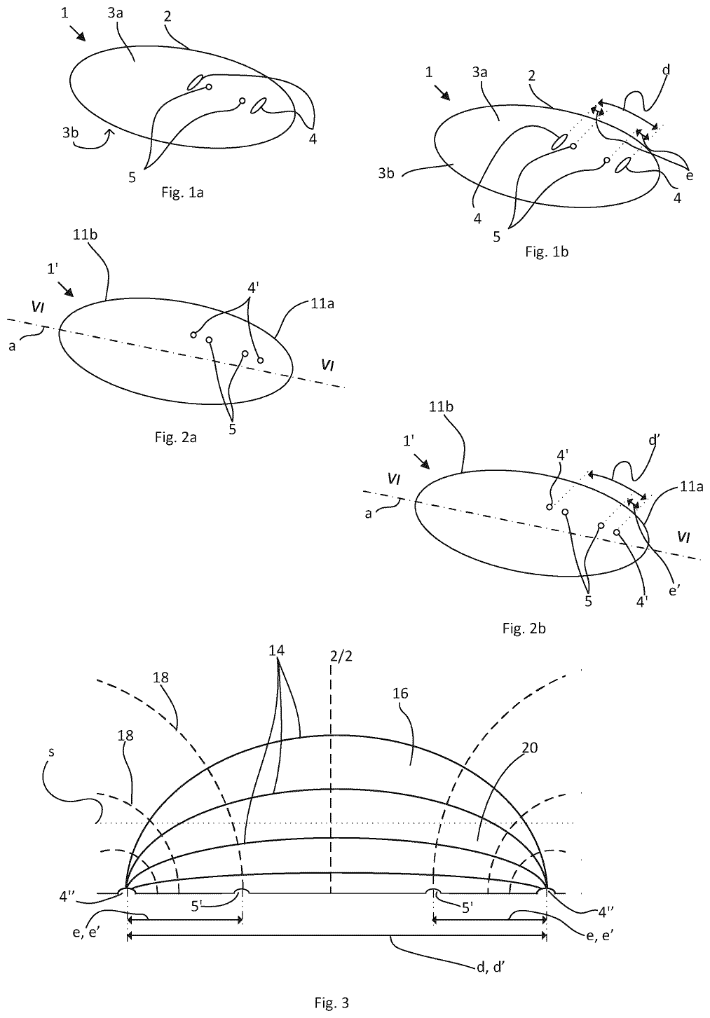

FIG. 1a illustrates an implantable sensor 1 comprising a housing 2 of double convex, ellipsoidal or ellipsoidal shape or just in general a rounded off and harmonic shape. The housing comprising a first part 3a and a second part 3b. The first part 3a, which could be considered the upper first part 3a, comprises two current injecting electrodes 4 and two voltage sensing electrodes 5. The current injecting electrodes 4 are configured to inject current and the voltage sensing electrodes 5 are configured to measure voltage and so determine impedance. The second part 3b, which could be considered the lower second part 3b, comprises fastening means (not shown) for fastening the implantable sensor 1 to tissue. The fastening means can be employed in the form of loops or hooks. The current injecting electrodes 4 and voltage sensing electrodes 5 are integrated in the housing 1 and arranged smooth and flush with an outer surface of the first part 3a. The current injecting electrodes 4 are of a longitudinal shape or ellipsoid shape, whereas the voltage sensing electrodes 5 are shown circular.

From FIG. 1a it can be seen that the voltage sensing electrodes 5 are arranged rather close to the current injection or current injecting electrodes 4. With reference to FIG. 1b the optimal distances between the pair of current injection electrodes 4 and the optimal distance between the voltage sensing electrodes 5 and the current injecting electrodes 4 will be further explained and discussed.

FIG. 1b shows the same or at least a very similar embodiment as FIG. 1a with a distance (d) indicated. The distance (d) describes and shows the distance as measured between the pair of current injecting electrodes 4. This distance (d) is optimally from 5 mm to 12 mm, preferably about 6 to 10 mm and more preferably about 7 to 9 mm. The distance (d) should be more than 4 mm for reasons explained in relation to FIG. 3.

Still referring to FIG. 1b, the centers of the current injection electrodes 4 and the voltage sensing electrodes 5 are arranged flush with the convex surface and the voltage sensing electrodes are arranged symmetrically between the current injection electrodes 4. The distance (e) between a first injection electrode to sensing electrode is the same as the distance (e) between the second voltage sensing electrode to second current injection electrode. This distance (e) is optimally chosen to be around 1 mm to 4 mm, preferably about 2 mm to 3 mm and actually depends on the distance (d). The distance e may be calculated using the following formula:

$$e < d / 2 ; whereby e \geq 1 mm .$$

thus (e) may preferably be smaller than half the distance (d) between the two injection electrodes.

The distances (d) and (e) may be measured on the surface of the housing 2 or they may be simplified and measured along a fictional straight line between the current injection electrodes.

The above-described distancing between the current injection electrodes 4 and the voltage sensing electrodes 5 works for any sensor shape and housing design. It is not limited to harmonic and rounded of shaped housings.

FIG. 2a illustrates a similar embodiment of an implantable sensor 1′ as FIG. 1 but in this case with circular current injecting electrodes 4′. All other features are the same or similar to the embodiment shown in FIGS. 1a and 1b. The circular current injecting electrodes 4′ work in a similar manner as the longitudinal current injection electrodes 4 shown with reference to FIGS. 1a and 1b.

In the embodiments shown in FIGS. 1a, 1b, 2a and 2b, the current injecting and voltage sensing electrodes 4, 4′, 5 are arranged in a row on the outer surface of the first part 3a with the two voltage sensing electrodes 5 being arranged in between the two current injecting electrodes 4, 4′.

FIG. 2a further illustrates the longitudinal axis (a) of the housing 2. In addition, FIG. 2a also illustrates the first half 11a and the second half 11b of the housing. The first half 11a and the second half 11b being defined by splitting the housing 2 in the middle as seen along the longitudinal axis (a) or split by a plane that extends perpendicular to the longitudinal axis (a) and which plane extends through a center of the housing 2.

FIG. 2b also illustrates the distances (d′) between the pair of injection electrodes 4′ and the distance (e′) between the first current injection electrode and the first voltage sensing electrode and the distance (e′), which is the same, between the second voltage sensing electrode and the second voltage sensing electrode.

The reason for the above distancing details becomes clear when viewing FIG. 3. FIG. 3 is a schematic representation that illustrates an electronic and geometric relationship between current trajectories and equipotential lines, in a cross-sectional view onto a plane that is cut through the electrodes 4, 4′, 5 and the longitudinal axis (a). FIG. 3 is to be understood to show illustratively the principle of a four-point measurement using a pair of current injecting electrodes 4″ and a pair of voltage sensing electrodes 5′. The shape of the different curves illustrated in FIG. 3 is not limiting nor is it directly binding, it is used for illustration. Further, the surface along which the electrodes 4″, 5′ are placed, is shown to be flat and not curved as in FIGS. 1a and 1b, for illustrative purposes. The principles described in relation to FIG. 3 also apply to curved surfaces, for example ellipsoidal surfaces. FIG. 3 illustrates two current injecting electrodes 4″ that extend electric field lines or current trajectories 14 from one current injecting electrode 4″ to the other current injecting electrode 4″ through organic tissue 16. The pair of current injecting electrodes 4″ are connected to a current source (not shown). FIG. 3 further illustrates a pair of voltage sensing electrodes 5′ arranged symmetrically in between the pair of current injecting electrodes 4″. The voltage sensing electrodes are designed to detect and measure impedance (voltage) between the equipotential lines 18 that define a volume (spheric surface) within the same voltage. The two voltage sensing electrodes 5′ are connected to a voltmeter and therewith the impedance in the tissue can be determined and measured.

FIG. 3 further illustrates the (fictional and simplified) line of the scar tissue(s) that is present when the sensor 1, 1′ is implanted in organic tissue of a living being. One can now understand why the distance (d), (d′) between the pair of current injecting electrodes 4″ is of importance. If this distance (d), (d′) is chosen to be too short then the current lines 14 will not extend into healthy tissue 16 and remain in the scar tissue 20 and therewith the voltage that is sensed between the equipotential lines 18 is only coming from the scar tissue 20 and the values will not be representative. If the distance (d), (d′) between the pair of current injecting electrodes 4″ is however chosen to be more than two times the thickness of the scar tissue 20 then the current trajectories 14 will start to extend into the healthy tissue 16 and the voltage sensing electrodes 5 can detect the voltage between equipotential lines that extend into the healthy tissue 16, as illustrated in FIG. 3. In FIG. 3, the distance (d), (d′) between the pair of injection electrodes 4″ is chosen to be about four times the thickness of the scar tissue 20, as measured perpendicular to the surface in which the electrodes 4″, 5 are arranged, thus about 8 mm.

The distance (d), (d′) can however not be chosen to be as great as possible either, since a great distance between the current injecting electrodes will raise other issues such as measurements in inhomogeneous tissue and artefacts in the measurement due to movement of the living being during measurement. That is the reason why the above given ranges are suitable.

Likewise FIG. 3 also illustrates the distance (e), (e′) between the first current injection electrode 4″ and the closest voltage sensing electrode 5. In the example of FIG. 3, this distance is about the thickness of the scar tissue 20, thus around 2 mm.

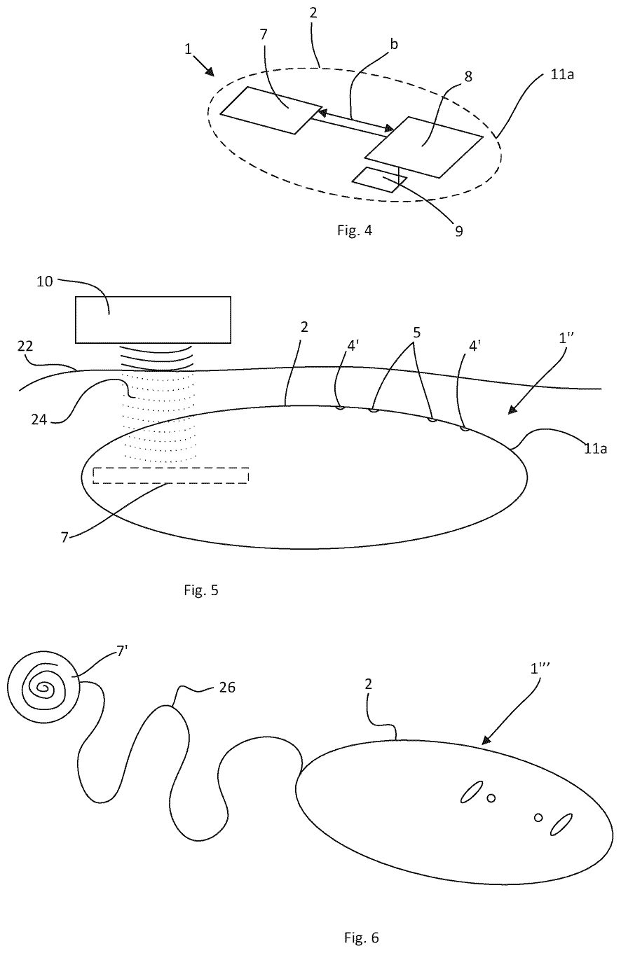

FIG. 4 illustrates the interior of the implantable sensor 1, comprising a coil 7, a circuit board 8 and a communication unit 9, which are electrically connected to one another. The housing 2 is shown in dashed lines to illustrate that it is omitted for illustrative purposes and so are the electrodes. The coil 7 is arranged next to the circuit board 8 but not overlapping. The two current injecting electrodes 4, 4′ and the two voltage sensing electrodes 5 are arranged on the first part 11a of the housing 2 that is arranged around the circuit board (not shown in FIG. 4 but c.f. FIG. 5). The two current injecting electrodes 4, 4′ and the two voltage sensing electrodes 5 are thus arranged above the circuit board 8 and not above the coil 7. The two current injecting electrodes 4, 4′ and the two voltage sensing electrodes 5 are further connected to the circuit board 8.

The positioning of the coil 7 away from the circuit board 8 by the distance (b) reduces the presence of Eddy Currents when the implantable glucose sensor 1, 1′, 1″, 1″ is powered by an external power source 10 as illustrated in FIG. 5.

FIG. 5 illustrates the measuring of the impedance of the tissue surrounding the sensor 1″ during powering of the implanted sensor 1″ via an external coil 10. Varying electromagnetic waves 24 originating from the external device induce a current in the coil 7, which current then powers the sensor 1″ for measuring impedance in the tissue/body fluid via the current injecting electrodes 4′ and the voltage sensing electrodes 5. The coil 7 is shown in dashed lines as FIG. 5 is actually a cross sectional view onto a cross section cut through the tissue of a patient showing the implanted sensor 1″ from the side. The external device 10 is arranged outside the body and skin 22 of the patient during powering of the sensor 1″. Although not shown in FIG. 5, the external device may be positioned adjacent the skin 22 during measurement.

Alternatively, to FIG. 5, the sensor 1″ may comprise a coil 7′ that is arranged outside the housing 2 of the sensor 1″ and connected to the electronics, such as the circuit board 8 of the sensor 1′″ via a cable as shown in FIG. 6. The reason for using a coil 7′ that is arranged separately from the housing 2 is that the coil can be placed close to the skin of a patient or living being so that it is easy for the external device 10 to power the sensor 1″ via the coil 7′ (c.f. FIG. 5).

The coil 7′ that is arranged outside the housing 2 may comprise means (not shown) for fixating it onto tissue within the body so that it cannot move or change its orientation after implantation.

The conductor 26 that is connecting the coil 7′ with the electronics of the sensor 1′″ may be a flexible cable coated with biocompatible material.

The embodiment shown in FIG. 6 can be embodied with the distancing of the current injecting and voltage sensing electrodes as described above, or it may be embodied without such a spacing of the electrodes and with another shaped housing 2. The idea or disclosure of having the coil 7′ arranged outside the housing 2 can be embodied with other sensors as well and is not limited to the disclosure herein and to implantable sensors designed to measure glucose.

FIG. 7 illustrates a measuring of the impedance of the tissue surrounding a sensor 1″ according to another embodiment and according to a third aspect described herein during powering using an external coil 10 of an external device, such as for example a mobile phone. FIG. 7 thereby shows a cross-sectional view cut through tissue and skin 22 and the implanted sensor 1″″ for illustrative purposes. Electromagnetic waves 24 originating from the external device induce a current in the coil 7″, which current then powers the sensor 1″″ for measuring impedance in the tissue/body fluid via the current injecting electrodes 4 and the voltage sensing electrodes 5. The current injecting electrodes 4 may be formed according to the embodiment shown in FIG. 1a or 2a. The housing 2″ of the sensor 1″″ is smaller than the housing 2, 2′ of the other embodiments. Whitin the housing 2″ the coil 7″ is connected to a ferrite sheet 32 and the ferrite sheet 32 is connected to the circuit board 8′. The ferrite sheet 32 may be glued, taped, bolted or otherwise fixed to the circuit board 8′. The coil 7″ may be glued or taped or bolted onto the ferrite sheet 32. The circuit board 8′, the ferrite sheet 32 and the coil 7″ may be arranged on top of one another and at least more or less congruent to one another as the cross section in FIG. 7 illustrates. The circuit board 8′ comprises a first part 8a, which first part 8a is flexible and can be bent to connected to the coil 7″. The first part 8a may even hold the coil 7″. The circuit board 8′ further comprises a second part 8b which is also a flexible part of the circuit board 8′, which second part 8b holds the current injecting electrodes 4 and the voltage sensing electrodes 5. The first part 8a, the second part 8b and the circuit board 8′ may be integrally formed. In an embodiment the coil 7″ may even be integrally formed with the circuit board 8′ and the coil 7″ may be arranged on the first flexible part 8a. The external device 10 is arranged outside the body and skin 22 of the patient during powering of the sensor 1″″. Although not shown in FIG. 5, the external device may be positioned adjacent the skin 22 during measurement.

As mentioned previously, the communication circuit 9 may be integrated in the circuit board 8′.

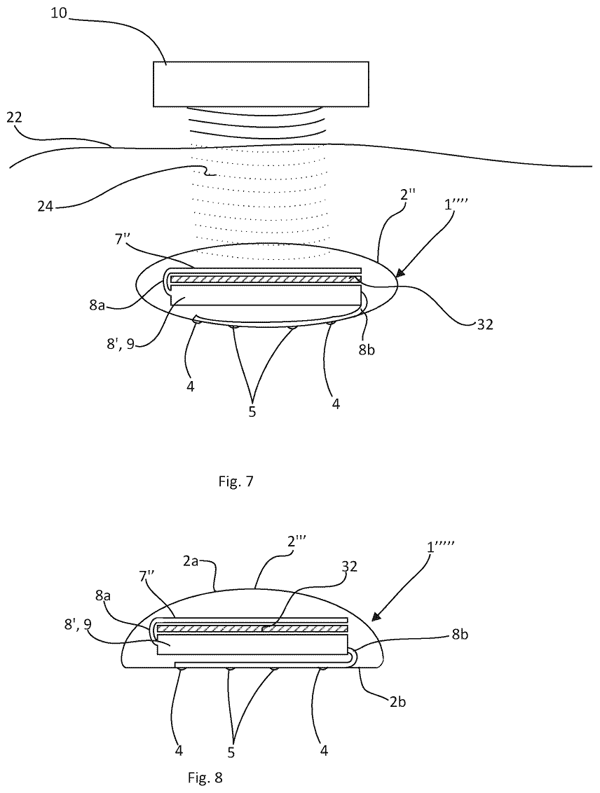

FIG. 8 illustrates a similar embodiment of the sensor 1″″″ as FIG. 7 but with another shape of the housing 2′″. The housing 2′″ of FIG. 8 is shaped half-convex with a convex side 2a and a flat side 2b. The voltage sensing electrodes 5 and the current injecting electrodes 4 are arranged on the flat side 2b of the housing 2′″. The buildup of the inside of the sensor 1″″ is otherwise very similar to FIG. 7, with the coil 7″ being separated from the circuit board 8′, 9 by a ferrite sheet 32. The circuit board 8′ comprises the first flexible part 8a and the second flexible part 8b while the current injecting electrodes 4 and the voltage sensing electrodes 5 are connected to and arranged or integrally formed on the second flexible part 8b.

The design of the housing 2′″ according to FIG. 8 may be advantageous since arranging the electrodes on a flat part or flat side 2b may facilitate assembly and design of the implantable sensor 1″″″. As explained previously the electrodes, thus the voltage sensing electrodes 5 and the current injecting electrodes 4 are again arranged on a side of the circuit board 8′ that is directed away from the coil 7″ and therewith on a side of the ferrite sheet 32 that is directed away from the coil 7″ for optimal performance and reduced or minimized magnetic, electromagnetic and/or electric disturbance during the measuring and powering of the implantable sensor 1″″.

Again, the shape of the current injecting electrodes may be according to FIG. 1a or according to 2a or according to the description herein. The electrodes shapes as described herein are not limited to a specific housing shape.

The invention has mainly been described above with reference to a few embodiments. However, as is readily appreciated by a person skilled in the art, other embodiment than the ones disclosed above are equally possible within the scope of the invention as defined by the appended patent claims.

1. An implantable sensor for determining glucose level based on electric impedance in tissue of a living being, the implantable sensor comprising:
a housing,
two current injecting electrodes for injecting current into the tissue,
two voltage sensing electrodes for measuring impedance in the tissue, whereby the two voltage sensing electrodes are arranged separately from the two current injecting electrodes and in between the current injecting electrodes,
a coil for powering the implantable glucose sensor via a power source,
a circuit board arranged within the housing, the circuit board being electrically connected to the two voltage sensing electrodes and the two current injecting electrodes and the coil, the coil being arranged on top of the circuit board,
a communication unit for transferring and receiving data packages, the communication unit being connected to the circuit board,
the housing comprising the two voltage sensing electrodes and the two current injecting electrodes on an outer surface,
characterized by further comprising a ferrite sheet arranged in between the coil and the circuit board, whereby the ferrite sheet has the same or a larger extension as the coil as seen in a plane defined by the circuit board.

2. The implantable sensor according to claim 1, wherein a distance between the two current injecting electrodes on the outer surface is in a range from 5 mm to 12 mm, 6 to 10 mm, or 7 to 9 mm.

3. The implantable sensor according to claim 1, wherein the ferrite sheet is arranged on one side of the circuit board and wherein the two current injecting electrodes and the two voltage sensing electrodes are arranged on an opposite side of the circuit board.

4. The implantable sensor according to claim 1, wherein the circuit board comprises at least two flexible integrated parts, a first flexible integrated part for connecting the coil to the circuit board and a second flexible integrated part for connecting the current injecting electrodes and the voltage sensing electrodes to the circuit board.

5. The implantable sensor according to claim 1, wherein the relationship between the distance between the two current injecting electrodes and a distance between one of the two voltage sensing electrodes and a closest injecting electrode and a distance between the other of the two voltage sensing electrodes and the other of the two current injecting electrodes, respectively follows the equation:
$$e < d / 2 and e \geq 1 mm .$$

6. The implantable sensor according to claim 1, wherein the voltage sensing electrodes are arranged in between the current injecting electrodes and wherein a distance between one of the two voltage sensing electrodes and a closest current injecting electrode and a distance between the other of the two voltage sensing electrodes and a closest current injecting electrode is in a range from 1 mm to 4 mm or 1.5 mm to 3 mm.

7. The implantable sensor according to claim 1, wherein the two current injecting electrodes comprise a first active area made of conductive material and wherein the voltage sensing electrodes comprise a second active area made of conductive material, wherein the first active area is bigger than the second active area.

8. The implantable sensor according to claim 1, wherein the first active area and the second active area are made of a bio-compatible conductive material such as gold, titan, platinum or any alloy thereof.

9. The implantable sensor according to claim 7, wherein the two current injecting electrodes have a longitudinal shape, such as for example an elliptic longitudinal shape or a rectangular longitudinal shape.

10. The implantable sensor according to claim 1, wherein the housing is shaped harmonic and rounded off and convex on at least one side.

11. The implantable sensor according to claim 1, wherein an other side of the housing is flat and wherein the voltage sensing electrodes and the current injecting electrodes are arranged on said other side, which is flat.

12. The implantable sensor according to claim 1, wherein the housing is of an ellipsoid shape.

13. The implantable sensor according to claim 1, wherein outer dimensions of the implantable sensor correspond to a length of 5 mm to 60 mm or 5 mm to 40 mm, a width of 5 mm to 25 mm or 11 mm to 15 mm, and a thickness of 1 mm to 15 mm or 2 mm to 5 mm.

14. The implantable sensor according to claim 1, wherein the sensor is an implantable glucose sensor.

15. The implantable sensor according to claim 1, wherein the two current injecting electrodes and the two voltage sensing electrodes are arranged along a straight line, wherein the straight line is straight in two dimensions, such as in a top-down view onto the implantable sensor.

16. The implantable sensor according to claim 1, wherein the voltage sensing electrodes are arranged symmetrically in between the injecting electrodes and wherein a distance between one of the two voltage sensing electrodes and a closest current injecting electrode and a distance between the other of the two voltage sensing electrodes and a closest current injecting electrode is in a range from 1 mm to 4 mm or 1.5 mm to 3 mm.

17. The implantable sensor according to claim 1, wherein the implantable sensor is an implantable glucose sensor and wherein a glucose concertation in the tissue is determined by correlating a measured impedance to a database containing impedance values and corresponding glucose concentrations.
